(19) United States
(12) Patent Application Publication
(10) Pub. No.: US 2011/0306875 A1
(43) Pub. Date: Dec. 15, 2011

(54) OPTICAL COHERENCE TOMOGRAPHY SYSTEM AND OPTICAL COHERENCE TOMOGRAPHY METHOD

(75) Inventors: Bjorn Fischer, Limbach-Oberfrohna (DE); Edmund Koch, Dresden (DE)

(73) Assignees: TECHNISCHE UNIVERSITAT DRESDEN, Dresden (DE); FRAUNHOFER-GESELLSCHAFT ZUR FÖRDERUNG DER ANGEWANDTEN FORSCHUNG E.V., München (DE)

(21) Appl. No.: 12/937,328

(22) PCT Filed: Apr. 11, 2008
(86) PCT No.: PCT/EP2008/002898

§ 371 Date: Jul. 18, 2011

(51) Int. Cl. A61B 6/03; G01B 11/24

(52) U.S. Cl. 600/425; 356/511

(57) ABSTRACT

The present invention relates to an optical coherence tomography system having an interferometer, in particular a Michelson interferometer, having a reference arm (R) for variable adjustment of an optical reference path length and having a measuring arm (M) in which an object (sample P) to be scanned can be disposed and/or is disposed in a sample volume (PV),

characterised in that
a focusing system (F) which is configured for focusing divergently incident light beams on a common point (target point Z) situated in the sample volume is disposed in the measuring arm between the beam splitter of the interferometer and the sample volume.

The present invention relates to an optical coherence tomography system and also to a corresponding optical coherence tomography method with which, by means of an interferometer, an object, in particular a biological sample, can be scanned.

Optical coherence tomography systems or coherence tomography methods are known already from the state of the art: the method known first and foremost from clinical fields, such as dermatology or ophthalmology, of optical coherence tomography (subsequently also abbreviated to OCT), generally uses radiation in the near infrared range between approx. 600 and 1,400 nanometres and enables the examination of semitransparent media, in particular biological samples. There serve as light sources of the methods or the systems, above all broadband superluminescent diodes with a short coherence length or also the quasi continuum pulsed laser (for example: femtosecond laser). The achievable resolving power of OCT thereby depends greatly upon the light source which is used and, according to the spectral width, is between 1 and 20 μm. The backscattered light contains information relating to the structure of the sample. This is superimposed with the light of a reference plane in an interferometer, in particular a Michelson interferometer, in a manner basically known to the person skilled in the art. The spectral intensity distribution at the output of the interferometer essentially corresponds to the Fourier transforms of the distribution of the scatter amplitude along the beam path.

An individual depth scan, as in ultrasonic methods, is hereby termed A-scan and contains the entire depth information at one point of the sample. By combining together a plurality of A-scans, the so-called B-image is obtained. In order to obtain three-dimensional data, for example an image stack can be produced from a plurality of B-images.

The method generally operates with 1,000 to 30,000 A-scans per second, from which between 2 and 50 B-images per second result.

The technology of OCT can quite generally also be used in other application fields, i.e. in application fields outside medicine: in particular OCT methods are used also in non-destructive testing of commercial materials.

One disadvantage of the optical coherence tomography systems or methods known from the state of the art is that, to date, examination of semitransparent materials behind greatly scattering or reflecting layers is not possible. Such layers represent a barrier for the wavelength ranges of the light source of the system or method which are used so that access to specific sample classes is not possible at the moment. In other words, sample areas which are situated behind a layer which is impenetrable for the wavelength used are not reached at the moment by an OCT examination.

Partly, the possibility exists in the state of the art of preparing the area to be examined to be exposed before the examination. However, this leads, in a not insignificant number of cases, to structural changes within the samples, which leads to the fact that the sample parameters detected by means of OCT methods can be used merely in a restricted fashion. All previously known methods (this applies for example also to so-called endoscopic OCT) hence require a direct access path without boundary layer to the area to be examined.

It is therefore the object of the present invention to develop the optical coherence tomography systems or methods known from the state of the art such that, with them, also those objects or samples which are present in the form of semitransparent materials behind greatly scattering and/or reflecting layers can be examined without preparation to be exposed or substantial structural changes.

The present object is achieved by an optical coherence tomography system according to claim 1 and an optical coherence tomography method according to claim 17. Advantageous embodiments of the system or method according to the invention are found respectively in the dependent patent claims. Uses according to the invention are described in patent claim 23.

Subsequently, the present invention is firstly described in general, then with the help of a special embodiment. The individual elements of the invention, as are described in the claims and in the description, need not be used in the special configuration shown in the embodiment, instead they can be used or applied, in the scope of the present invention, based on the expert knowledge of the person skilled in the art, also in differently configured configurations and/or arrangements. In particular, the present invention is explained with reference to a simple frequency-based OCT system (English: Fourier-domain OCT). Instead of using a rotatable deflecting unit, thereby shown, for one-dimensional lateral scanning of the object in order to obtain a B-scan, the device according to the invention can also be produced correspondingly in an OCT system with a single-line detector or a two-dimensional array detector, in which one plane is exposed and evaluated within the sample or the entire sample at the same time. Which components must thereby be exchanged in detail in order to enable such a parallel OCT is basically known to the person skilled in the art (see for example the article “Optical Coherence Tomography—Principles and Applications”, A. F. Fercher et al., Rep. Prog. Phys. 66 (2003), pp. 239-303).

The basis of the present invention is a special lens system (subsequently also termed focusing system F) which is introduced into the measuring arm of the optical coherence tomography system between the beam splitter of the interferometer and the sample volume (in which the sample to be scanned is disposed). This focusing system is configured such that, with it, divergent light beams which impinge on the focusing system and/or which are produced in the interferometer, in particular in the measuring arm and/or by the focusing system itself, are focused on precisely one common point (subsequently: target point Z) situated in the sample volume. The angle of incidence is subsequently viewed, unless otherwise stated, as that direction in the measuring arm which extends from the beam splitter of the interferometer towards the sample (correspondingly, the reverse direction, i.e. the direction in which the light reflected and/or scattered at the sample extends back again to the beam splitter and finally towards the detector, is termed emergence direction unless otherwise stated.

In the sample volume, the sample to be scanned, in particular a biological object, is then disposed. In order to enable scanning of such samples which have a reflecting and/or non-transparent boundary layer for the incident radiation, a perforation, in particular in the form of a hole, is introduced into the boundary layer of the sample however before the sample is scanned. The sample is then disposed in the sample volume such that the above-described target point falls precisely in the hole or the perforation of the sample or such that the target point comes to lie just behind the hole in the boundary layer, i.e. within the sample. In this way, the entire light used for scanning the sample is focused through a narrow or small opening within the boundary layer of the sample into the sample interior (which is semitransparent for the incident light). Also the light components reflected or scattered in this interior of the sample, which are then directed in the beam exit direction through the focusing system and detected in the normal manner with OCT by means of a detector, are beamed through the small opening introduced in the sample in the reverse direction.

Advantageously, the focusing system according to the invention is configured such that the light beams emerging divergently from a source point Q (a point which lies in the beam incidence direction after the beam splitter of the interferometer and in front of the sample) are focused on the target point with the help of the focusing system.

In order to achieve such focusing, two planar-convex lenses which are directed with their curvature towards each other, can be used in the measuring arm or, alternatively thereto, two achromatic lenses. In addition, the use of meniscus lenses and/or partial spherical lenses configured essentially in the shape of a hemisphere (aplanatic lenses) is possible. The precise construction of the focusing system according to the invention is described subsequently in even more detail.

The great advantage of the optical coherence tomography system or method according to the invention is that a large part of the sample spectrum, which has not been accessible to date for OCT, can be made accessible with the help of the construction according to the invention and with the help of a perforation in the screening boundary surface of the sample. The described method or the described optical coherence tomography system therefore enables examination of semitransparent media through a small opening: keyhole OCT.

The present invention is now described subsequently with reference to a detailed embodiment.

There are shown

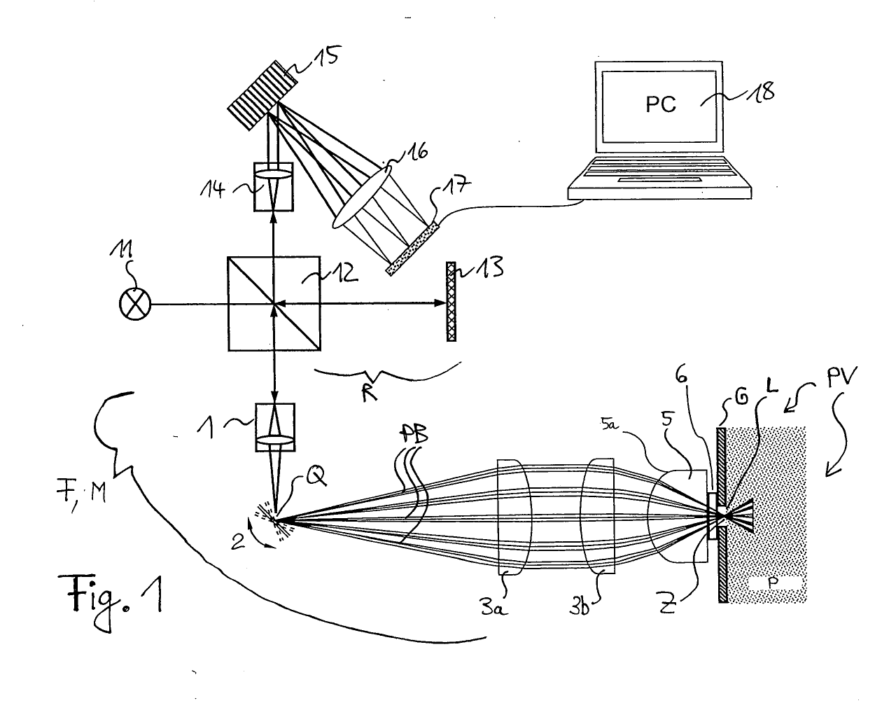

FIG. 1 the construction of the optical coherence tomography system according to the embodiment and

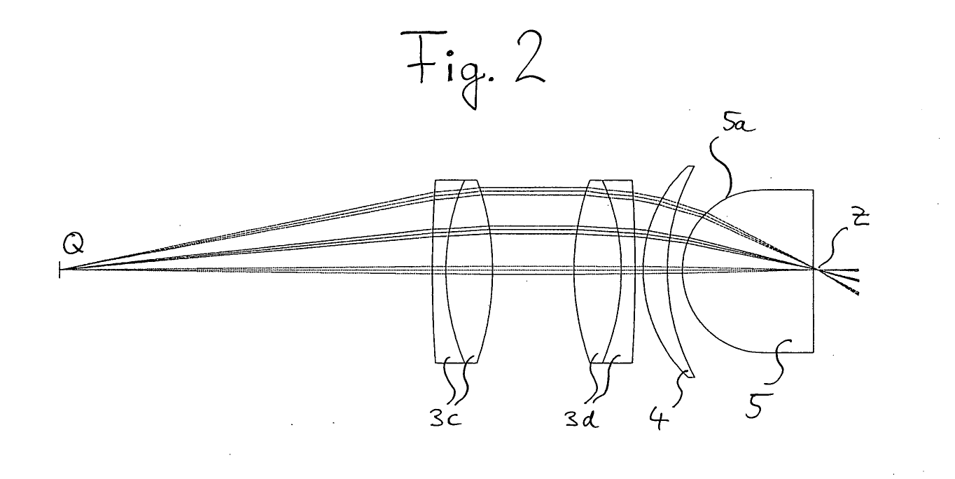

FIG. 2 an improved lens system for use in the embodiment according to FIG. 1.

FIG. 1 shows the basic construction of an optical coherence tomography system according to the invention. The basic components 11 to 18 of an interferometer, here a Michelson interferometer, are basically known to the person skilled in the art from prior art: the light of a white light source 11 is directed towards a semipermeable beam splitter 12 and, from there, is directed towards a mirror 13 in the reference arm R, on the one hand, and, on the other hand, into the measuring arm M. As a result of the fact that the light reflected at the mirror 13 comes to interfere with the sample light in the beam splitter 12 and is cast for evaluation by the collimator 14 via the dispersing grating 15 and an imaging lens system, in particular a focusing lens system 16, on to the detector 17 (to which a computer system 18 is connected for evaluation), the depth information can be obtained from the spectrum by means of Fourier transformation. How this takes place in detail is known to the person skilled in the art from prior art, therefore it is not dealt with here further with respect to the evaluation components. Alternatively, also a time-domain OCT can be used, in which the grating and the detector line can be dispensed with, however instead a length change to the reference arm must be possible.

The optical coherence tomography system according to the invention now differs from this system known from the state of the art by the construction of the measuring arm. The measuring arm M, in the illustrated example, is, in the beam entrance direction (i.e. viewed from the beam splitter 12 in the direction of the sample P), constructed as follows: the light reflected from the beam splitter into the measuring arm is firstly beamed by a focusing unit 1 onto a deflecting mirror 2 which is disposed in the beam path after the focusing unit 1 and is rotatable or pivotable about an axis perpendicular to the illustrated plane. The focusing unit 1 is hereby constructed and disposed such that the light coming from the beam splitter 12 is focused onto a point on the surface of the deflecting mirror 2. This point is subsequently termed also source point and provided with the reference Q.

The light emanating from the surface point of the mirror 2 or from the source point Q is now reflected in the beam path of the measuring arm M onto an aplanatic lens 3 which consists of two planar-convex lenses 3a and 3b disposed in succession in the beam path. The two planar-convex lenses 3a and 3b are thereby disposed in the beam path such that they are directed with their curvature towards each other. After these two planar-convex lenses 3a, 3b, there follows as further element of the focusing system F a lens (partial spherical lens 5, subsequently termed also in a simplified manner as hemispherical lens) which is configured essentially as a spherical section (here: essentially with a hemispherical configuration; the spherical section can however also include a larger angle range than 180°). The surface configured essentially as part of a spherical surface or the curved surface 5a of this lens 5 is thereby directed in the beam exit direction or towards the two planar-convex lenses 3a, 3b. The surface 5a of the lens 5 is configured as aplanatic surface (see H. Haferkorn, “Optik”, 3^{rd} edition, p. 318 ff.). Abutting directly on the surface, opposite the surface 5a, of the partial spherical lens 5 which is configured as planar surface, a layer 6 made of immersion liquid (frequently: immersion oil) is disposed, directly abutting on which the sample P is located with its boundary surface G orientated towards the immersion layer 6 (the sample P, G is hereby disposed in the sample volume PV of the measuring arm).

The above-described embodiment with the optical elements 1, 2, 3a, 3b, 5, and 6 is a possible embodiment of the principle underlying the present invention: quite in general, this optical system must be configured hence such that a beam path is produced which images the beams emanating from points of the mirror 2 into the hole L as free of errors as possible (see subsequent paragraph), the angle range of the sample portions detected in the sample behind the hole being intended to be as large as possible. A simple 1:1 imaging hereby achieves for instance an angle range of 20° which is reduced by the refraction (as a function of the concrete configuration of the sample) in the sample volume once again by the factor of the refractive index of the sample. The partial spherical lens 5 increases this angle range in combination with the immersion liquid by the factor n² (with n as the refractive index of the glass of the lens which is approx. 1.5). Each further meniscus lens hereby again produces a factor of n.

In the illustrated sample P, a small perforation in the form of a hole L is now introduced into the boundary surface G orientated towards the focusing system F thereof (comprising the elements 1, 2, 3a, 3b, 5 and 6). This perforation or this hole L makes it possible for the incident light from the measuring arm M to penetrate into the interior of the sample and not to be reflected on the exterior boundary surface G. The focusing system F or the individual elements 1, 2, 3a, 3b, 5 and 6 thereof are now configured and disposed such that all the light beams directed onto the aperture of the lens system 3a, 3b with the help of the rotatable deflecting mirror 2, are focused by the optical system 3a, 3b, 5 and 6 onto a single common point, the target point Z. This point is disposed (or the sample P is disposed) such that it comes to lie precisely in the perforation or in the hole L. For this reason, it is made possible for all the light beams (represented by the beams PB) which emanate divergently from the source point Q and are collected by the aperture of the lens system 3a, 3b, 5, 6 to penetrate through the perforate L into the actual semitransparent interior of the sample P. In this interior, the light is then reflected and/or scattered according to the conditions or structures prevailing there so that light components reflected or scattered back in the direction of the entrance point can pass through the perforation L and can approach the beam exit-side path via the focusing system F, the beam splitter 12 and also the further optical elements 14-16 towards the detection system 17, 18. It is hereby crucial that all the divergently emanating beams from the source point Q are focused by the lens system 3-6 onto the target point Z lying in the perforation (in order to achieve good transverse resolution, the focus Z of the beam can be placed also just behind the perforation or behind the boundary layer in the interior of the sample P).

The sample beams or sample ray bundles PB are hence focused onto the target point Z with the help of the lens system according to the invention (focusing system F) such that examination of the sample P through a small cylindrical opening L in the external boundary layer G of the sample becomes possible. For this purpose, the light is focused onto the deflecting unit 2 for beam deflection. The sample beam diverging after passage through the hole L into the sample (the divergence is achieved here such that the deflecting mirror 2 is pivoted by a specific angle value so that the aperture of the subsequent optical elements is exploited as completely as possible; this can however also be produced by simultaneous illumination of the entire aperture) then allows contact-free examination of the structures and materials situated behind the non-transparent layer G, analogously to conventional OCT measuring technology. By varying the reference arm length (e.g. by displacing the mirror 13 along the incident beam), the measuring range of the OCT system can be adapted to the measuring volume.

In the present case, the centre of rotation of the beam deflection (the point Q) is hence placed by the lens system F on the entrance opening L so that the sample beam PB, in every position of the deflecting unit 2 passes through the hole L in the sample.

In order to achieve optimum transverse resolution in the sample P, the focus of the sample beam (or the target point Z) can alternatively also be placed at the level of the boundary surface not precisely in the hole but just behind the perforation L, i.e. already in the interior of the sample P. The spacing from the perforation L to the target point Z should thereby be less than 10 mm.

This can be achieved for example by the focal point of the white light source 11 being placed not directly on the surface of the deflecting unit 2 but displaced by a small distance behind this surface of the beam deflecting unit 2. It must hereby be ensured that the beam cross-section of the sample beam PB then also passes through the hole L in the sample and hence substantial components of the sample beam PB are not reflected at the circumferential hole edges.

In the present example, an immersion lens 5, 6 is inserted between the second planar-convex lens 3b, orientated towards the sample P, and the external boundary surface G of the sample, which immersion lens is configured in the incident radiation direction firstly from the partially spherical lens 5 and then the immersion layer made of immersion liquid 6 disposed thereon. This has the advantage that, if a sample with a refractive index which is >1 is situated behind the perforation L, the scanning region is enlarged. It must hereby be ensured that both the partially spherical lens 5 and the external boundary surface G or the sample P are in optical contact with the immersion layer 6 (avoidance of air gaps etc.). In order to reduce the imaging error of the spherical aberration and to increase the scanning region further, the opening L in the boundary layer G is placed in one of the aplanatic points of the spherical surface 5a of the lens 5.

Introduction of the minimum opening L in the outer skin of many systems P is in many cases not critical: the perforation L serves for the duration of the examination as a window and can subsequently be closed again. Both technical and biological samples P which to date eluded examination in the state of the art are hence accessible.

In an alternative variant, the system shown in FIG. 1 can be configured to be pivotable in the illustrated plane for further enlargement of the scanning range (i.e. the volume detected within the sample P). The pivot axis is then directed perpendicular to the illustrated plane (i.e. parallel to the axis of rotation of the unit 2). This can be achieved for example by the sample volume (including the sample P) being configured to be pivotable together with the immersion layer 6 and the lens 5 about the centre of curvature of the spherical surface of the lens 5 in the plane represented in FIG. 1. As a result, the object region scanned behind the perforation L in the sample P is correspondingly enlarged. As an alternative thereto, also the entire system consisting of the interferometer components 11-17 and the elements 1, 2, 3a and 3b can however also be configured to be pivotable in the mentioned plane (pivot axis through the centre of curvature of the spherical surface 5a). Further pivotable configuration possibilities exist (thus basically also the unit 3a, 3b could be configured rotatably about a spatial centre disposed in the region of the elements 5, 6 or G so that, when using a larger pivot angle of the deflecting mirror 2, a larger range can also be scanned within the sample volume P as a result).

In the presented example, the optical coherence tomography system according to the invention is explained with reference to a simple frequency range coherence tomography system. Of course, the present invention can however also be produced within the scope of a time-domain coherence tomography system. Furthermore, in particular also the use of the present invention is possible in a manner directly comprehensible to the person skilled in the art in a parallel OCT coherence tomography system. Such a parallel system has a single-line, multiple-line or an array detector. The latter can be produced in particular by means of a commercial CCD camera. Such parallel OCT systems detect one line (in the illustrated plane) or the entire image of the object P in parallel (in the former case, also the beam is scanned in one direction by the deflection unit 2 in order to detect the entire image. In the latter case, the light of the sample is directed towards an array detector and either the principle of time-domain OCT or the principle of swept source OCT must be applied). It is hereby essential that, instead of using a deflecting mirror 2, all the represented partial bundles of the sample beam PB are focused simultaneously onto the sample P, i.e. the entire aperture of the elements 3a, 3b is illuminated at the same time.

With respect to technical equipment, this can be achieved for example by a powerfully focusing optical system 1 (e.g. a microscope objective).

As is known to the person skilled in the art, if the entire region to be configured, here the entire aperture, is illuminated in the illustrated plane, in the case of parallel line-OCT systems, then the entire aperture is observed also at the same time: different angle ranges in the sample beam PB are sent for this purpose to different elements of the detector line and an array spectrometer is used instead of the detector line 17. In the case of OCT systems which detect the entire image in a parallel manner (perpendicular to the illustrated plane), a two-dimensional array detector (surface detector) is used instead of the illustrated detector 17. In this case, either the principle of time-domain OCT can be applied, i.e. the length of the reference arm is slowly changed and the detector is read out every time or a single frequency light source (swept source) is used instead of the white light source 11, with which light source the entire light spectrum to be used (for example the frequencies of 600-800 nm or 1,100 to 1,300 nm) can be run through in succession. Hence, the wavelength of the radiated light is slowly changed and the sample light is sent to the array detector (CCD camera).

FIG. 2 shows an example of an improved lens system which can be used as part of the focusing system of the embodiment shown in FIG. 1. The optical system shown in FIG. 2 thereby replaces the elements 3a, 3b and 5 shown in FIG. 1. In the case of this improved lens system, there are disposed, in the incident beam path (i.e. viewed from the source point Q towards the target point Z), the following elements in succession: first achromatic lens 3c, second achromatic lens 3d, meniscus lens 4 and partial spherical lens 5 with aplanatic surface 5a. The curves of the two achromatic lenses are hereby directed towards each other, as in the case shown in FIG. 1. The curves of the meniscus lens 4 are directed in the direction of the subsequent partial spherical lens 5. With the illustrated lens system, larger angle ranges can be imaged with good imaging quality than with the lens system 3a, 3b and 5 shown in FIG. 1. The achromatic lenses 3c, 3d are used instead of the simple planar-convex lenses 3a and 3b in order to reduce the spherical aberration. In addition to the aplanatic surface 5a in the lens 5 which increases the numerical aperture NA, the meniscus lens 4 is used for repeated increase in the numerical aperture NA. The meniscus lens 4 is hereby configured such that, on the surface of the meniscus lens orientated towards the lens 5, no refraction takes place (the beams pass here precisely perpendicularly through the surface), whereas, on the left surface (the surface orientated towards the achromatic lens 3d) of this lens, the beams are refracted without imaging errors because of the shaping of this surface. Calculation of the required radii and distances can be effected by the person skilled in the art according to the formulae described in Haferkorn, “Optik”, 3^{rd} edition, p. 318 ff. and chapter 4.3.3. The concrete production hereby depends for example upon focal distance and diameter of the achromatic lenses.

The previously described principle shown in FIG. 2 can also be used repeatedly to increase the numerical aperture NA: with every further aplanatic lens which is disposed between partially spherical lens and achromatic lenses, the NA is increased by the factor n, n being the refractive index of such a lens which will normally be around 1.5. As emerges from the above description, the radii of the spherical surfaces must be staggered such that the beams pass perpendicularly through the surface, respectively on the side orientated towards the achromatic lenses, whereas, on the side orientated towards the partially spherical lens, the beams are refracted such that the numerical aperture continues to be increased without undesired spherical aberration resulting.

Hence a plurality of meniscus lenses can be used; it is also possible to use a plurality of achromatic lenses, if necessary together with a plurality of meniscus lenses.

By means of further lenses in the illustrated optical structure which can involve in particular scattering lenses made of a material with high dispersion, the colour error of the system can be reduced further.

The claimed invention is:

1.-26. (canceled)

27. An optical coherence tomography system comprising:
an interferometer, including:
a beam splitter;
a reference arm for variable adjustment of an optical reference path length; and
a measuring arm in which an object to be scanned can be disposed in a sample volume;
a focusing system, configured for focusing divergent light beams onto a common target point situated in the sample volume, the focusing system being disposed in the measuring arm between the beam splitter and the sample volume,
wherein the focusing system is configured for focusing light beams onto the target point, which light beams emerge divergently from a source point which, viewed in the incident beam path of the measuring arm, is situated after the beam splitter,
wherein the focusing system, viewed in the incident beam path of the measuring arm includes, after the beam splitter, two planar-convex lenses that are directed with their curvature towards each other, or two achromatic lenses that are directed with their greater curvature towards each other, and
wherein the focusing system is configured such that bundles of beams that impinge at different angles of incidence on the two planar-convex lenses or achromatic lenses are capable of being focused onto the common target point.

28. The optical coherence tomography system according to claim 27, wherein the focusing system, viewed in the incident beam path of the measuring arm, includes, after the beam splitter and after the two planar-convex lenses or achromatic lenses, a meniscus lens, curves of which, viewed in the incident beam path of the measuring arm, point towards a beam exit side.

29. The optical coherence tomography system according to claim 28, wherein the meniscus lens is configured such that, viewed in the incident beam path of the measuring arm, no light refraction takes place on the beam exit side of said meniscus lens.

30. The optical coherence tomography system according to claim 28, wherein the focusing system includes, in the measuring arm and, viewed in the incident beam path of the measuring arm, after the beam splitter and after the two planar-convex lenses or achromatic lenses, a partially spherical lens including a partially spherically configured surface on a beam entrance side when viewed in the incident beam path of the measuring arm.

31. The optical coherence tomography system according to claim 30 wherein at least one of:
the partially spherically configured surface of the partially spherical lens is configured as an aplanatic surface;
the surface of the partially spherical lens situated opposite the partially spherically configured surface is configured as a flat surface; and/or in that
the partially spherical lens is configured in the form of an immersion objective; or
an immersion liquid is disposed between the partially spherical lens and the sample volume.

32. The optical coherence tomography system according to claim 28, wherein the focusing system, viewed in the incident beam path of the measuring arm, includes, after the beam splitter, a plurality of meniscus lenses with different radii configured such that no light refraction takes place on the beam exit side thereof.

33. The optical coherence tomography system according to claim 27 wherein a refractive index for at least one lens included in the system is in the range between 1.4 and 1.8.

34. The optical coherence tomography system according to claim 27 wherein the focusing system, viewed in the incident beam path of the measuring arm, includes, after the beam splitter and before the two planar-convex lenses or achromatic lenses, a rotatable and/or pivotable deflecting unit, with which the incident beams can be directed onto different partial regions of the aperture of the two planar-convex lenses or achromatic lenses.

35. The optical coherence tomography system according to claim 34 wherein the focusing system, viewed in the incident beam path of the measuring arm, includes, after the beam splitter and before the deflecting unit, a focusing element that is configured for focusing the incident light beams onto the deflecting unit.

36. The optical coherence tomography system according to claim 35 wherein the focal point of the focusing element onto the deflecting unit corresponds to the source point.

37. The optical coherence tomography system according to claim 35 wherein the focusing system is configured such that, during deflection of the incident beams by the deflecting unit bundles of beams that impinge at different angles of incidence on the two planar convex lenses or achromatic lenses are focused onto the target point via an aperture of the two planar-convex lenses or achromatic lenses.

38. The optical coherence tomography system according to claim 27, wherein the focusing system is configured such that at least one of (1) essentially the entire aperture of the two planar-convex lenses or achromatic lenses is capable of being illuminated at the same time in at least one direction or (2) the sample light from different angle segments is capable of being guided onto different detector elements of a detector.

39. The optical coherence tomography system according to claim 27 and including at least one element that is rotatable and/or pivotable about a stationary spatial point relative to the interferometer, which is disposed in a stationary manner in space or to one or more components of the interferometer and relative to the sample volume, which is disposed in a stationary manner in space,
wherein the interferometer, together with at least part of the focusing system, is rotatable and/or pivotable about a stationary spatial point, relative to the sample volume, or
wherein the sample volume is rotatable and/or pivotable about a stationary spatial point relative to at least part of the interferometer.

40. The optical coherence tomography system according to claim 27, configured as an optical time-domain coherence tomography system or as an optical frequency range coherence tomography system.

41. The optical coherence tomography system according to claim 27 configured as a parallel optical coherence tomography system using a single-line, multiple-line, or an array detector, as a polarization-sensitive optical coherence tomography system, as an optical Doppler coherence tomography system, and/or as an endoscopic optical coherence tomography system.

42. The optical coherence tomography system according to claim 41 wherein the optical coherence tomography system includes an array detector includes a tunable single frequency light source with variable frequency for the emitted light.

43. An optical coherence tomography method, comprising the following steps:
disposing an object to be scanned in a sample volume in a measuring arm of an interferometer having a reference arm for variable adjustment of an optical reference path length and having said measuring arm;
producing divergent light beams;
disposing, before scanning the object, in the measuring arm between a beam splitter of the interferometer and the sample volume, a focusing system that is configured for focusing the produced divergent light beams onto a common target point situated in the sample volume; and
introducing, before scanning the object, a perforation into an external boundary layer of the object and disposing the object in the sample volume such that the target point is situated in this perforation or, viewed from the focusing system, immediately behind it within the object.

44. The optical coherence tomography method according to claim 43 comprising scanning the object using an optical coherence tomography system comprising:
an interferometer, including:
a beam splitter;
a reference arm for variable adjustment of an optical reference path length; and
a measuring arm in which an object to be scanned can be disposed in a sample volume;
a focusing system, configured for focusing divergent light beams onto a common target point situated in the sample volume, the focusing system being disposed in the measuring arm between the beam splitter and the sample volume,
wherein the focusing system is configured for focusing light beams onto the target point, which light beams emerge divergently from a source point which, viewed in the incident beam path of the measuring arm, is situated after the beam splitter,
wherein the focusing system, viewed in the incident beam path of the measuring arm includes, after the beam splitter, two planar-convex lenses that are directed with their curvature towards each other, or two achromatic lenses that are directed with their greater curvature towards each other, and
wherein the focusing system is configured such that bundles of beams that impinge at different angles of incidence on the two planar-convex lenses or achromatic lenses are capable of being focused onto the common target point.

45. The optical coherence tomography method according to claim 43, comprising using the perforation having an average diameter of 10 μm to 1 mm.

46. The optical coherence tomography method according to claim 43 comprising using a spacing between the perforation and the target point that is on average less than 10 mm.

47. The optical coherence tomography method according to claim 43 comprising scanning a biological sample as the object and comprising introducing a perforation in a light-reflecting external boundary layer of the sample before the scanning is performed.

48. The optical coherence tomography method according to claim 47 comprising placing the perforation in the boundary layer in an aplanatic point of a hemispherical lens.
